(19) United States
(12) Patent Application Publication
(10) Pub. No.: US 2022/0396615 A1
(43) Pub. Date: Dec. 15, 2022

(54) ANTI-HUMAN IL6 MONOCLONAL ANTIBODIES, PREPARATION METHOD THEREFOR AND USE THEREOF

(71) Applicant: NANJING GENSCRIPT BIOTECH CO.,LTD., Jiangsu (CN)

(72) Inventors: Liusong Yin, Jiangsu (CN); Feng Lin, Jiangsu (CN); Lei Wang, Jiangsu (CN); Yunyun Xu, Jiangsu (CN); Li Ding, Jiangsu (CN)

(21) Appl. No.: 16/770,308

(22) PCT Filed: Dec. 5, 2018
(86) PCT No.: PCT/CN2018/119353

§ 371 Date: Jun. 5, 2020

(30) Foreign Application Priority Data
Dec. 5, 2017 (CN) 201711269749.6

(51) Int. Cl. C07K 16/24; A61P 35/00

(52) U.S. Cl. CPC C07K 16/248; A61P 35/00; C07K 2317/92; C07K 2317/51; C07K 2317/515

(57) ABSTRACT

The present invention provide anti-human IL6 monoclonal antibodies, amino acid sequences of the variable region of the heavy chain and of the variable region of the light chain thereof, and encoding nucleotide sequences thereof. The present invention also provides a method for preparing the anti-human IL6 monoclonal antibodies and use of the anti-human IL6 monoclonal antibodies in the preparation of an antitumor drug. The present anti-human IL6 monoclonal antibodies can inhibit cell proliferation by blocking an IL6 signal pathway, thereby achieving the purpose of tumor immunotherapy.

### FIELD OF THE INVENTION

The present invention belongs to the field of tumor immunotherapy and molecular immunology, particularly relates to anti-human IL6 monoclonal antibodies. The present invention further relates to a method for preparing the anti-human IL6 monoclonal antibodies, and use of the anti-human IL6 monoclonal antibodies.

### BACKGROUND OF THE INVENTION

Immune system is a host defense system. In order to function normally, an immune system must be able to detect the invasion of foreign pathogens sensitively, and distinguish them from the healthy tissues of an organism itself. The immune system of a vertebrate is a functional system consisting of a variety of organs, tissues, cells and molecules, and is the most effective mechanism for an organism to defend against the invasion of foreign substances; these immune organs, tissues, cells and molecules cooperate with each other and restrict each other to achieve a balance, so as to protect the organism from external infestation and maintain the balance in the body (Janeway et al., Immunology: The Immune System in Health and Disease. New York: Garland Science, 2005). Such mutual cooperation and mutual restriction require the coordination of numerous immune checkpoint proteins and cytokines, wherein stimulatory immune checkpoint proteins enhance the defense response of an immune system, and inhibitory immune checkpoint proteins control an overly strong immune system to prevent the generation of an autoimmune response. Carcinogenesis is resulted from the loss of normal cell regulatory functions in normal somatic cells and the accumulation of gene mutations to a certain degree. In tumor treatment, tumor immunotherapy is widely used, and has become one of the most important means for tumor treatment. Tumor immunotherapy enables human T cells to better recognize cancerous cells by enhancing the immune function in human body, thereby killing cancer cells. For example, Pembrolizumab, an antibody against inhibitory immune checkpoint protein PD1, and Ipilimumab, an antibody against CTLA4, have been approved for use in the treatment of a variety of cancer indications. However, due to tumor heterogeneity and inter-tumor differences of tumor immunity, these immune checkpoint proteins-based antibody drugs have very limited clinical benefits.

Interleukins, the most important category of cytokines, have a variety of immunomodulary functions of directing the maturation, differentiation, migration and adherence of cells in immune system. In tumorigenesis, these cytokines directly stimulate immune effectors and stromal cells in tumor sites and enhance tumor cell recognition of cytotoxic effector cells (Yoshimoto et al., Immunotherapy 5:825-844, 2009). Recently, some studies show that interleukins participate in a lot of tumor-associated molecule mechanisms and have been used to develop many cytokine-based cancer treatment methods. However, tumor cells can escape from immune system by many methods, including establishment of a safe tumor microenvironment, secretion of cytokines and anti-inflammatory cytokines that suppress immune system, and recruitment or conversion of inflammatory cells that suppress immune responses, including regulatory T (Treg) cells, bone marrow derived suppressor cells (MDSC) and dendritic cells (DC) (Stewart et al., Cancer Metastasis Rev. 30:125-140, 2011). Interleukin family members are present in the tumor microenvironment and interact with various biomolecules such as cancer stem cells, microRNAs, and epithelial mesenchymal cells. Therefore, based on the principle that interleukins affect mechanisms of tumor formation, high-efficient cancer immunotherapy can be developed. Over the past decade, some relevant therapies including interleukin 2 (IL2), interleukin 6 (IL6), interleukin 7 (IL7), interleukin 12 (IL12), interleukin 18 (IL18) and interleukin 21 (IL21) have entered the clinical trial stage in patients.

Interleukin 6 (IL6) has a variety of functions in vivo, among which this factor can cause imbalance and secretion of a variety of inflammatory substances of regulatory T cells (Treg). Studies have shown that evaluated serum IL6 concentration in patients is closely associated with tumor staging of various cancers (e.g., multiple myeloma, non-small cell lung cancer, colorectal cancer, renal cancer, prostate cancer, breast cancer and ovarian cancer) and survival of patients. Moreover, IL6 can induce epithelial-mesenchymal transition in breast cancer cells to generate CD44 positive cells with the characteristics of stem cells. There is evidence showing that the IL6 level in tumor-associated endothelial cells is directly associated with the tumorigenicity of tumor stem cells (Int. J. Mol. Sci. 16:1691-1710, 2015). Therefore, it is a potential treatment strategy for cancer to block IL6 signaling. The anti-human IL6 antibody Siltuximab from Johnson & Johnson Pharmaceuticals was approved by the U.S. Food and Drug Administration in 2014 for use in the treatment of Castleman's disease and now is undergoing a Phase II clinical trial directed to multiple myeloma. However, so far, there are no anti-human IL6 antibodies that are commercially available for tumor immunotherapy. Moreover, different anti-human IL6 monoclonal antibodies have different degrees of side effects, including the possibility of inducing immunogenicity in some patients, and different IL6 monoclonal antibodies have different degrees of developability. Therefore, there is still a need to develop new functional antibodies capable of blocking IL6 signaling, which should have higher affinity, specificity, functionality, and diversity.

### SUMMARY OF THE INVENTION

In one aspect, the present invention provides an anti-human IL6 monoclonal antibody, comprising a heavy chain variable region and a light chain variable region, wherein the heavy chain variable region comprises HCDR1, HCDR2 and HCDR3, and the light chain variable region comprises LCDR1, LCDR2 and LCDR3, wherein the HCDR1, HCDR2, HCDR3, LCDR1, LCDR2 and LCDR3 are selected from one of the following combinations:

(a)
the amino acid sequence of HCDR1 is
RYWMH;
the amino acid sequence of HCDR2 is
YINPITGYTENNQKFKD;
the amino acid sequence of HCDR3 is
GIRGFTY;
the amino acid sequence of LCDR1 is
RASENVDNSDNSFMH;
the amino acid sequence of LCDR2 is
RASNLDS;
the amino acid sequence of LCDR3 is
QQTNEAPLT;
(b)
the amino acid sequence of HCDR1 is
NYWMH;
the amino acid sequence of HCDR2 is
YVIPSTGYTDYNQSFKD;
the amino acid sequence of HCDR3 is
LLPGFAY;
the amino acid sequence of LCDR1 is
RSSQSLVDSNGNTYLH;
the amino acid sequence of LCDR2 is
KVSNRFS;
the amino acid sequence of LCDR3 is
SQSTHVPPT;
(c)
the amino acid sequence of HCDR1 is
NYWMH;
the amino acid sequence of HCDR2 is
YIDPRTASIYYNQKFKD;
the amino acid sequence of HCDR3 is
ILYGKYDV;
the amino acid sequence of LCDR1 is
RSSQSLVDSNGNTYLH;
the amino acid sequence of LCDR2 is
KVSNRFS;
the amino acid sequence of LCDR3 is
SQSTHVPPT;
(d)
the amino acid sequence of HCDR1 is
DAWMD;
the amino acid sequence of HCDR2 is
EIRSKTYHPATYYTKSVRG;
the amino acid sequence of HCDR3 is
PRYYGGYFDY;
the amino acid sequence of LCDR1 is
RASESVDNYGMSFMN;
the amino acid sequence of LCDR2 is
TASNQGS;
the amino acid sequence of LCDR3 is
QQSKEVPYT;
(e)
the amino acid sequence of HCDR1 is
NYIIH;
the amino acid sequence of HCDR2 is
AIYPGNGDTSYSQKFKD;
the amino acid sequence of HCDR3 is
GDAGYSAWFAY;
the amino acid sequence of LCDR1 is
SASESVDSYGNNFMH;
the amino acid sequence of LCDR2 is
LASKLES;
the amino acid sequence of LCDR3 is
QQNNEDPLT;
(f)
the amino acid sequence of HCDR1 is
SHTVS;
the amino acid sequence of HCDR2 is
KMWSNGDTDYDSAIRS;
the amino acid sequence of HCDR3 is
YYFSSYGGGYFDY;
the amino acid sequence of LCDR1 is
RASKSVSTYMH;
the amino acid sequence of LCDR2 is
SASNLES;
the amino acid sequence of LCDR3 is
QQSDELPDT;
and
(g)
the amino acid sequence of HCDR1 is
SFPMA;
the amino acid sequence of HCDR2 is
TISPSGGTSYSRDSVKG;
the amino acid sequence of HCDR3 is
ERIYNTYFDY;
the amino acid sequence of LCDR1 is
LPSEDISSDLA;
the amino acid sequence of LCDR2 is
NANTLPN;
the amino acid sequence of LCDR3 is
QQYDSYPYT.

In more particular embodiments, the amino acid sequences of the heavy chain variable region and light chain variable region of the anti-human IL6 monoclonal antibody are selected from one of the following combinations:

the heavy chain variable region comprising the amino acid sequence set forth in SEQ ID NO: 1, and the light chain variable region comprising the amino acid sequence set forth in SEQ ID NO: 3;

the heavy chain variable region comprising the amino acid sequence set forth in SEQ ID NO: 5, and the light chain variable region comprising the amino acid sequence set forth in SEQ ID NO: 7;

the heavy chain variable region comprising the amino acid sequence set forth in SEQ ID NO: 9, and the light chain variable region comprising the amino acid sequence set forth in SEQ ID NO: 11;

the heavy chain variable region comprising the amino acid sequence set forth in SEQ ID NO: 13, and the light chain variable region comprising the amino acid sequence set forth in SEQ ID NO: 15;

the heavy chain variable region comprising the amino acid sequence set forth in SEQ ID NO: 17, and the light chain variable region comprising the amino acid sequence set forth in SEQ ID NO: 19;

the heavy chain variable region comprising the amino acid sequence set forth in SEQ ID NO: 21, and the light chain variable region comprising the amino acid sequence set forth in SEQ ID NO: 23; and

the heavy chain variable region comprising the amino acid sequence set forth in SEQ ID NO: 25, and the light chain variable region comprising the amino acid sequence set forth in SEQ ID NO: 27.

In preferred embodiments, the dissociation constant KD between the anti-human IL6 monoclonal antibody and IL6 is less than 10 nM. In more preferred embodiments, the dissociation constant KD between the anti-human IL6 monoclonal antibody and IL6 is less than 1 nM.

In an embodiment, the anti-human IL6 monoclonal antibody inhibits the promotion of TF-1 cell proliferation by IL6.

In another aspect, the present invention provides an isolated polynucleotide sequence that encodes the anti-human IL6 monoclonal antibody.

In a particular embodiment, the polynucleotide comprises a heavy chain variable region coding sequence that encodes the heavy chain variable region of the anti-human IL6 monoclonal antibody, and a light chain variable region coding sequence that encodes the light chain variable region of the anti-human IL6 monoclonal antibody, wherein the heavy chain variable region coding sequence and the light chain variable region coding sequence are selected from one of the following combinations:

the heavy chain variable region coding sequence comprising the nucleotide sequence set forth in SEQ ID NO: 2, and the light chain variable region coding sequence comprising the nucleotide sequence set forth in SEQ ID NO: 4;

the heavy chain variable region coding sequence comprising the nucleotide sequence set forth in SEQ ID NO: 6, and the light chain variable region coding sequence comprising the nucleotide sequence set forth in SEQ ID NO: 8;

the heavy chain variable region coding sequence comprising the nucleotide sequence set forth in SEQ ID NO: 10, and the light chain variable region coding sequence comprising the nucleotide sequence set forth in SEQ ID NO: 12;

the heavy chain variable region coding sequence comprising the nucleotide sequence set forth in SEQ ID NO: 14, and the light chain variable region coding sequence comprising the nucleotide sequence set forth in SEQ ID NO: 16;

the heavy chain variable region coding sequence comprising the nucleotide sequence set forth in SEQ ID NO: 18, and the light chain variable region coding sequence comprising the nucleotide sequence set forth in SEQ ID NO: 20;

the heavy chain variable region coding sequence comprising the nucleotide sequence set forth in SEQ ID NO: 22, and the light chain variable region coding sequence comprising the nucleotide sequence set forth in SEQ ID NO: 24; and

the heavy chain variable region coding sequence comprising the nucleotide sequence set forth in SEQ ID NO: 26, and the light chain variable region coding sequence comprising the nucleotide sequence set forth in SEQ ID NO: 28.

In another aspect, the present invention provides an expression vector comprising the above polynucleotide.

In another aspect, the present invention provides a host cell comprising the above expression vector.

In preferred embodiments, the host cell is HEK293-6E cell.

In another aspect, the present invention provides a method for preparing an anti-human IL6 monoclonal antibody, comprising transfecting a competent cell with the above expression vector, and culturing the cell.

In another aspect, the present invention provides use of the anti-human IL6 monoclonal antibody, the polynucleotide, the expression vector, the host cell in the preparation of an anti-tumor medicament.

In preferred embodiments, the tumor is selected from multiple myeloma, non-small cell lung cancer, colorectal cancer, renal cancer, prostate cancer, breast cancer and ovarian cancer.

In another aspect, the present invention provides an antitumor pharmaceutical composition, comprising an effective amount of the anti-human IL6 monoclonal antibody, and a pharmaceutically acceptable carrier.

In a further aspect, the present invention provides a method for preparing an anti-human IL6 monoclonal antibody, comprising:

1) immunizing a mouse with human IL6, and generating an immune response against human IL6 in the mouse;

2) fusing the spleen cells obtained from the mouse with myeloma cells, and screening the resulting hybridoma cells, to obtain a positive parent clone that specifically recognizes human IL6;

3) subcloning the positive parent clone, to obtain a stable hybridoma cell line;

4) subjecting the hybridoma cell line to gene sequencing, to obtain the variable region coding sequences of an anti-human IL6 antibody; and

5) using the variable region coding sequences to produce a recombinant antibody, thereby obtaining a functional anti-human IL6 monoclonal antibody.

The anti-IL6 monoclonal antibodies as provided in the present invention have high affinity and high specificity for IL6, and can recognize different epitopes of IL6, respectively, can stimulate or inhibit IL6 downstream pathway, activate or inhibit secretion of cytokines by T cells, for example, inhibit the proliferation of TF-1 cells by inhibiting IL6 downstream pathway. Therefore, the functional monoclonal antibodies against IL6 as provided in the present invention, can inhibit cell proliferation by blocking IL6 signal pathway, and further achieve the purpose of tumor immunotherapy.

### BRIEF DESCRIPTION OF THE DRAWINGS

FIG. 1 shows the ELISA titer test results of sera from mice immunized with human IL6.

FIG. 2 shows the ELISA titer test results of sera from rats immunized with human IL6.

FIG. 3 shows the curve of the specific binding of the anti-IL6 monoclonal antibodies of the present invention to IL6 recombinant protein.

FIG. 4 shows the curve of the specific inhibition of TF-1 cell proliferation by the anti-IL6 monoclonal antibodies of the present invention.

FIG. 5 shows the measurement result of the affinity of the anti-IL6 monoclonal antibodies of the present invention for IL6 protein.

### DETAILED DESCRIPTION OF THE INVENTION

Unless otherwise specified, the technical and scientific terms used in the present invention have the same meanings as generally understood by those skilled in the art.

As used herein, the term “antibody” generally refers to an immunoglobulin molecule, which is usually a tetramer composed of two identical heavy chains and two identical light chains connected to each other via disulfide bonds. According to the differences in the conservation of the amino acid sequences, heavy chains and light chains are divided into a variable region (V) at the amino terminal and a constant region (C) at the carboxy terminal. In each of the variable regions of heavy chains and light chains, there are three local regions, which have a higher degree of variation in the amino acid composition and arrangement order and are the key positions for the binding of an antibody to an antigen, and therefore are also called complementarity determining regions (CDR). In the context, the three heavy chain complementarity determining regions are called HCDR1, HCDR2 and HCDR3, respectively, and the three light chain complementarity determining regions are called LCDR1, LCDR2 and LCDR3, respectively. The variable regions of one heavy chain and one light chain interact with each other to form an antigen binding site (Fv). Depending on the amino acid sequences of the heavy chain constant regions, antibodies can be divided into different classes. There are five main classes of intact antibodies: IgA, IgD, IgE, IgG and IgM, and some of them can be further divided into subclasses, such as IgG1, IgG2, IgG3, IgG4, IgA and IgA2. For different classes of immunoglobulins, their subunit structures and three-dimensional conformations are known in the art. The present invention is intended to include any of the aforementioned classes or subclasses of antibodies.

As used herein, the term “antibody” is also intended to encompass its digestive fragments or functional variants, for example, antibody fragments capable of binding to IL6 or parts thereof, including, but not limited to Fab (e.g., those obtained by papain digestion of antibodies), F(ab′)2 (e.g., those obtained by pepsin digestion), Fv or scFv (e.g., those obtained by molecular biotechnology).

As used herein, the term “monoclonal antibody” refers to an antibody that is homogeneous and is directed only against a certain specific antigenic epitope. In contrast to common polyclonal antibody preparations which typically include different antibodies against different antigenic determinants (epitopes), each monoclonal antibody is directed against a single antigenic determinant on the antigen. The term “monoclonal” refers to the homogenous characteristic of antibodies and should not be explained as antibodies produced by any specific method. The monoclonal antibodies of the present invention are preferably produced by recombinant DNA method, or by screening methods described elsewhere herein.

As used herein, the term “an isolated polynucleotide” refers to a polynucleotide in a state that is not naturally occurring in nature, including polynucleotides isolated from nature (including organisms) by biological techniques, and also including artificially synthesized polynucleotides. An isolated polynucleotide can be genomic DNA, cDNA, mRNA or other synthetic RNA, or a combination thereof. The present invention provides multiple nucleotide sequences encoding heavy chain variable regions and light chain variable regions of anti-human IL6 monoclonal antibodies. It should be noted that those skilled in the art, based on the degeneracy of codons, can design nucleotide sequences that are not completely identical to the nucleotide sequences provided above, but encode the same amino acid sequences, according to the amino acid sequences of heavy chain variable regions and light chain variable regions provided herein. These altered nucleotide sequences are also included in the scope of the present invention.

When relating to polynucleotide, the term “vector” used herein refers to any molecule (e.g., nucleic acids, plasmids, or viruses, etc.) for transferring nucleotide encoding information to a host cell. The term “expression vector” or “expression cassette” refers to a vector suitable for expression of a gene of interest (a nucleotide sequence to be expressed) in a host cell, which generally comprises the elements such as the gene of interest, a promoter, a terminator, and a marker gene.

As used herein, the term “host cell” refers to a cell that has been or can be transformed with a nucleic acid sequence and thereby expresses the selected gene of interest. The term includes the progeny of parent cells, no matter whether the progeny is identical to the original parent cells in morphology or genetic composition, as long as the selected gene of interest is present in the progeny. Commonly used host cells include bacteria, yeasts, mammalian cells, etc.

As used herein, the term “transfection” refers to the uptake of foreign or exogenous DNA by the cell. This technique can be used to introduce one or more exogenous DNA parts into a suitable host cell. Cells can be induced by physicochemical methods (such as by calcium chloride treatment) so that they are in a physiological state most suitable for uptaking and accommodating foreign DNA, i.e. “competent”.

When a pharmaceutical composition is mentioned, the term “an effective amount” used herein refers to an amount that can lead to a function or activity in human and/or animal and can be accepted by human and/or animal. The term “a pharmaceutically acceptable carrier” refers to a carrier for administration, including various excipients, diluents, buffers and the like, which are suitable for administration to human and/or animal without excessive adverse side effects, and meanwhile are suitable for maintaining the activity of the drug or active agent contained therein.

Some aspects of the present invention will be described in detail by combining the following examples. Unless otherwise specified, the methods and materials in the Examples described below are commercially available conventional products.

### EXAMPLES

### Example 1: Obtainment of Human IL6 Hybridoma Cell Line

1) Animal Immunization

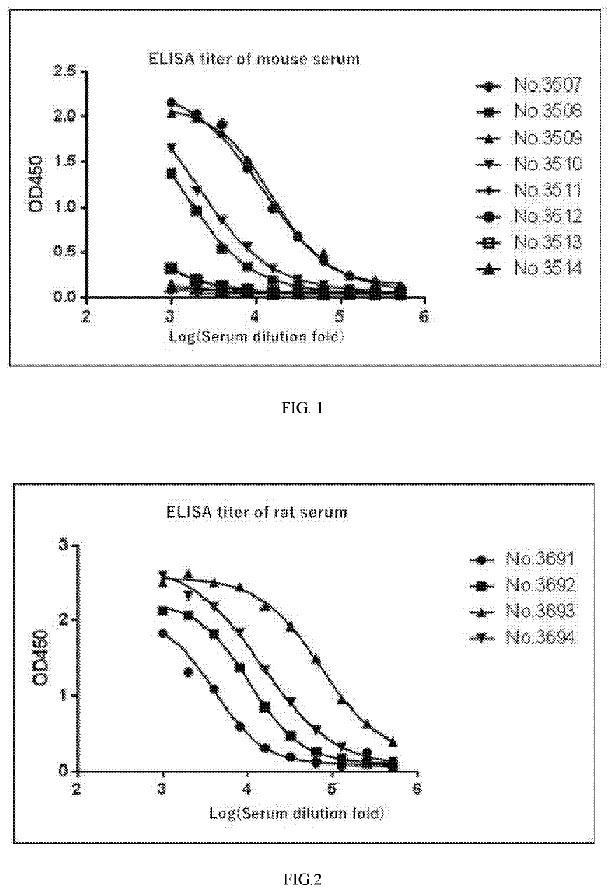

Tag-free human IL6 recombinant protein (GenScript, Cat. No. Z03034) was used as antigen. Female Balb/c and C57bl/6 mice and Wistar rats were subcutaneously immunized with a 1:1 emulsion of 200 μl Freund's complete adjuvant (Sigma-Aldrich) containing 50 μg IL6 protein. Then, a 1:1 emulsion of Freund's incomplete adjuvant (Sigma-Aldrich) containing 25 μg IL6 was injected intraperitoneally/subcutaneously alternately every two weeks up to three times, thereby boosting the mice. Four days before myeloma fusion, a mouse (#3509) and a rat (#3693) showing the highest antibody titer (see FIG. 1 and FIG. 2, the antibody titer was determined by using serum ELISA method) were boosted by intraperitoneal administration of 25 μg IL6 (without adjuvant).

2) Hybridoma Fusion and Screening

The spleen was extracted and homogenized to produce a single-cell suspension, and meanwhile a single-cell suspension of myeloma cell (SP2/0) was prepared. 8.9×10⁷ spleen cells and 4.1×10⁷ SP2/0 mouse myeloma cells were fused by electrofusion. The fused cells were re-suspended in 100 ml DMEM/10% FBS medium containing hybridoma cell selectors thymidine, hypoxanthine and aminopterin, and a volume of 100 μl was transferred to a 96-well plate with a pipette. The plate was incubated at 37° C. in 6% CO₂. After incubation for 7 days, the presence of antibodies against IL6 was detected by using the ELISA binding assay described below.

ELISA binding assay: antibodies in the supernatant were evaluated for the binding ability to IL6 by indirect ELISA. The ELISA plate (Nunc) was coated with 0.5 μg/ml recombinant IL6 protein in PBS at 100 μl/well at 4° C. overnight. The plate was washed with PBS-T (0.05% Tween) and blocked with PBST containing 1% BSA at 200 μl/well at 37° C. for 0.5 h. The blocking solution was then discarded, and 100 μl hybridoma cell culture supernatant was added to each plate. The incubation was then performed for 1 h at room temperature. The plate was washed with PBST for three times and was incubated with horseradish peroxidase-conjugated goat anti-mouse IgG (Fab-specific) (GenScript) at 100 μl/well at 37° C. for 0.5 h. The plate was washed with PBST for five times, and TMB chromogenic solution (GenScript) was then added. Incubation was performed at room temperature in dark for 15 min. The reaction was stopped by adding 50 μl 1M HCl stop solution (Sigma). The plate was read at 450 nm by a Microplate Reader.

3) Hybridoma Subcloning

Subcloning was performed by limiting dilution method. A hemocytometer was used and cells were subjected to serial dilution in DMEM/10% FBS medium containing hybridoma cell selectors thymine, hypoxanthine and aminopterin to determine the number of cells until the cell density reached 5-15 cells/ml. For each hybridoma, 200 μl cell solution was pipetted into 96 wells at a density of 1-3 cells/well. After culturing the cultures at 37° C. in 5% CO₂ for 1 week, the supernatant was subjected to the ELISA binding assay described above to evaluate the presence of antibodies against IL6.

### Example 2: Sequencing of Variable Regions of Monoclonal Antibodies and Recombinant Production of Antibodies

After identifying the subtypes of antibodies in the hybridoma cell culture supernatant with a rapid ELISA mouse antibody subtype identification kit (Clonotyping System-HRP, SouthernBiotech), total RNA was extracted from 3×10⁶-5×10⁶ hybridoma cells by using TRIzol (Ambion), and was reverse-transcribed into cDNA by using antibody subtype-specific primers and universal primers (PrimeScript™ 1stStrand cDNA Synthesis Kit, Takara). The mouse immunoglobulin heavy and light chain V-region fragments were then amplified by RACE PCR (GenScript), and the resulting PCR fragments were subcloned into pMD18-T vector system (Takara), and the inserted fragments were sequenced using vector-specific primer pairs. The unique V-region nucleotide/amino acid sequences of the clones 8H10B7E6, 23A9H3, 29D6B5, 41A10B8, 49F10H6, 53A2F9 (rat), and 61G1D8 (rat) were finally obtained. In the amino acid sequences of the heavy chain and light chain, the CDR1 regions are underlined with solid line, the CDR2 regions are underlined with dotted line, and the CDR3 regions are underlined with wavy line.

The amino acid sequence of heavy chain variable region of 8H10B7E6: SEQ ID NO: 1

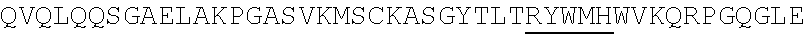

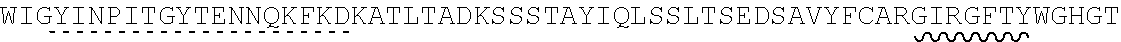

LVTVSA

The nucleotide sequence of heavy chain variable region of 8H10B7E6: SEQ ID NO: 2

CAGGTCCAGCTTCAGCAGTCTGGGGCTGAACTGGCAAAACCTGGGGCCTC
AGTGAAGATGTCCTGCAAGGCTTCTGGCTACACCTTAACTAGGTACTGGA
TGCACTGGGTAAAACAGAGGCCTGGACAGGGTCTGGAATGGATTGGATAC
ATTAATCCTATCACTGGTTATACTGAGAACAATCAGAAGTTCAAGGACAA
GGCCACATTGACTGCAGACAAGTCCTCCAGCACAGCCTATATACAACTGA
GCAGCCTGACATCTGAGGACTCTGCAGTCTATTTCTGTGCAAGAGGGATA
CGGGGGTTTACTTACTGGGGCCATGGGACTCTGGTCACTGTCTCTGCA

The amino acid sequence of light chain variable region of 8H10B7E6: SEQ ID NO: 3

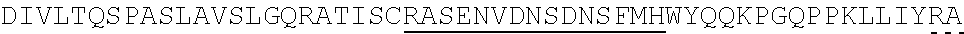

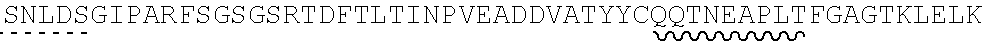

The nucleotide sequence of light chain variable region of 8H10B7E6: SEQ ID NO: 4

GACATTGTGCTGACCCAATCTCCAGCTTCTTTGGCTGTGTCTCTAGGGCA
GAGGGCCACCATATCCTGCAGAGCCAGTGAGAATGTTGATAACTCTGACA
ATAGTTTTATGCACTGGTACCAACAGAAACCAGGACAGCCACCCAAACTC
CTCATCTATCGTGCATCCAACCTAGATTCTGGGATCCCTGCCAGGTTCAG
TGGCAGTGGGTCTAGGACAGACTTCACCCTCACCATTAATCCTGTGGAGG
CTGATGATGTTGCAACCTATTACTGTCAGCAAACTAATGAGGCTCCGCTC
ACGTTCGGTGCTGGGACCAAGCTGGAGCTGAAA

The amino acid sequence of heavy chain variable region of 23A9H3: SEQ ID NO: 5

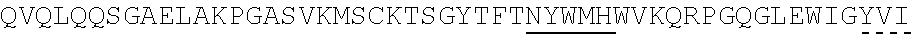

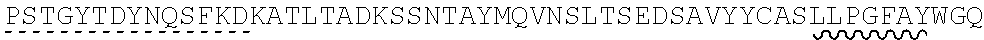

GTLVTVSA

The nucleotide sequence of heavy chain variable region of 23A9H3: SEQ ID NO: 6

CAGGTCCAGCTTCAGCAGTCTGGGGCTGAACTGGCAAAACCTGGGGCCTC
AGTGAAGATGTCCTGCAAGACTTCTGGCTACACCTTTACTAACTACTGGA
TGCACTGGGTAAAACAGAGGCCTGGACAGGGTCTGGAGTGGATTGGATAC
GTTATTCCTAGCACTGGTTATACTGACTACAATCAGAGTTTCAAGGACAA
GGCCACATTGACTGCAGACAAATCCTCCAACACAGCCTACATGCAAGTGA
ACAGCCTGACATCTGAGGACTCTGCAGTCTATTACTGTGCAAGCCTACTG
CCAGGTTTTGCTTACTGGGGCCAAGGGACTCTGGTCACTGTCTCTGCA

The amino acid sequence of light chain variable region of 23A9H3: SEQ ID NO: 7

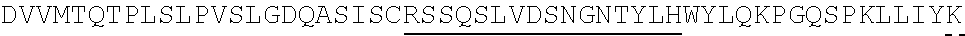

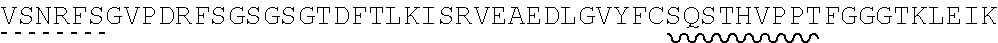

The nucleotide sequence of light chain variable region of 23A9H3: SEQ ID NO: 8

GATGTTGTGATGACCCAAACTCCACTCTCCCTGCCTGTCAGTCTTGGAGA
TCAAGCCTCCATCTCTTGCAGATCTAGTCAGAGCCTTGTGGACAGTAATG
GAAACACTTATTTACATTGGTACCTGCAGAAGCCAGGCCAGTCTCCAAAG
CTCCTGATCTACAAAGTTTCCAACCGATTTTCTGGGGTCCCAGACAGGTT
CAGTGGCAGTGGATCAGGGACAGATTTCACACTCAAGATCAGCAGAGTGG
AGGCTGAGGATCTGGGGGTTTATTTCTGCTCTCAAAGTACACATGTTCCT
CCGACGTTCGGTGGAGGCACCAAGCTGGAAATCAAA

The amino acid sequence of heavy chain variable region of 29D6B5: SEQ ID NO: 9

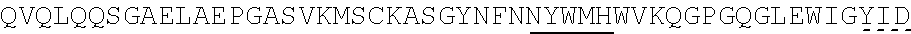

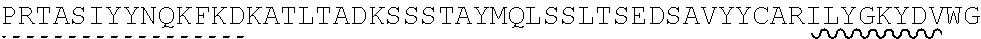

AGTTVIVSS

The nucleotide sequence of heavy chain variable region of 29D6B5: SEQ ID NO: 10

CAGGTCCAGCTTCAGCAGTCTGGGGCTGAACTGGCAGAACCTGGGGCCTC
AGTGAAGATGTCCTGCAAGGCTTCTGGCTACAACTTTAATAACTATTGGA
TGCACTGGGTAAAACAGGGGCCTGGCCAGGGTCTGGAATGGATTGGATAC
ATTGATCCTAGGACTGCTTCTATTTATTACAATCAGAAGTTCAAAGACAA
GGCCACATTGACTGCAGACAAATCCTCCAGCACAGCCTACATGCAACTGA
GCAGCCTGACATCTGAAGACTCTGCAGTCTATTACTGTGCAAGAATCCTC
TATGGTAAATATGATGTCTGGGGCGCAGGGACCACGGTCATCGTCTCCTC
A

The amino acid sequence of light chain variable region of 29D6B5: SEQ ID NO: 11

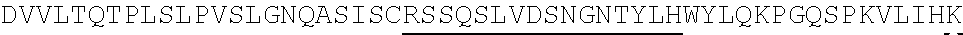

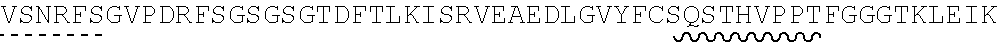

The nucleotide sequence of light chain variable region of 29D6B5: SEQ ID NO: 12

GATGTTGTGCTGACCCAAACTCCACTCTCCCTGCCTGTCAGTCTTGGAAA
TCAAGCCTCCATCTCTTGCAGATCTAGTCAGAGCCTTGTAGACAGTAATG
GAAACACCTATTTACATTGGTACCTGCAGAAGCCAGGCCAGTCTCCAAAG
GTCCTGATCCACAAAGTTTCCAACCGATTTTCTGGGGTCCCAGACAGGTT
CAGTGGCAGTGGATCAGGGACAGATTTCACACTCAAGATCAGCAGAGTGG
AGGCTGAGGATCTGGGAGTTTATTTCTGCTCTCAAAGTACACATGTTCCT
CCGACGTTCGGTGGAGGCACCAAGCTGGAAATCAAA

The amino acid sequence of heavy chain variable region of 41A10B8: SEQ ID NO: 13

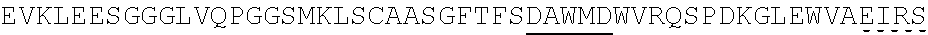

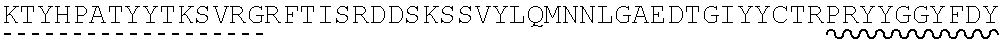

WGQGTTLTVSS

The nucleotide sequence of heavy chain variable region of 41A10B8: SEQ ID NO: 14

GAAGTGAAACTTGAGGAGTCTGGAGGAGGCTTGGTGCAACCTGGAGGATC
CATGAAACTCTCTTGTGCTGCCTCTGGATTCACTTTTAGTGACGCCTGGA
TGGACTGGGTCCGCCAGTCTCCAGACAAGGGGCTTGAGTGGGTTGCTGAA
ATTAGAAGCAAAACTTATCATCCTGCAACATACTATACTAAGTCTGTGAG
AGGGAGGTTCACCATCTCAAGAGATGATTCCAAAAGTAGTGTCTACCTGC
AGATGAACAACTTAGGAGCTGAAGACACTGGCATTTATTACTGTACCAGG
CCGAGATACTACGGGGGTTACTTTGACTACTGGGGCCAAGGCACCACTCT
CACAGTCTCCTCA

The amino acid sequence of light chain variable region of 41A10B8: SEQ ID NO: 15

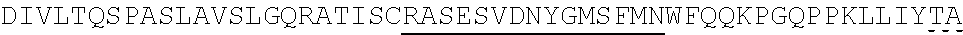

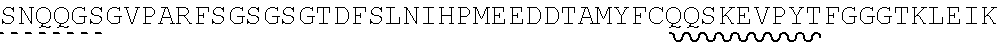

The nucleotide sequence of light chain variable region of 41A10B8: SEQ ID NO: 16

GACATTGTGCTGACCCAATCTCCAGCTTCTTTGGCTGTGTCTCTAGGGCA
GAGGGCCACCATCTCCTGCAGAGCCAGCGAAAGTGTTGACAATTATGGCA
TGAGTTTTATGAACTGGTTCCAACAGAAACCAGGACAGCCACCCAAACTC
CTCATCTATACTGCTTCCAACCAAGGATCCGGGGTCCCTGCCAGGTTTAG
TGGCAGTGGGTCTGGGACAGACTTCAGCCTCAACATCCATCCTATGGAGG
AGGATGATACTGCAATGTATTTCTGTCAACAAAGTAAGGAGGTCCCGTAT
ACGTTCGGAGGGGGGACCAAGCTGGAAATAAAA

The amino acid sequence of heavy chain variable region of 49F10H6: SEQ ID NO: 17

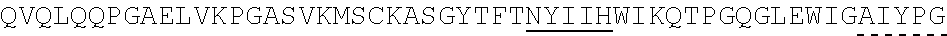

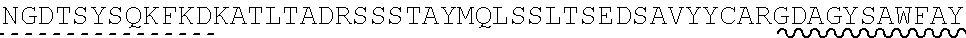

WGQGTLVTVSA

The nucleotide sequence of heavy chain variable region of 49F10H6: SEQ ID NO: 18

CAGGTGCAACTGCAGCAGCCTGGGGCTGAACTGGTGAAGCCTGGGGCCTC
AGTGAAGATGTCCTGCAAGGCTTCTGGCTACACATTTACCAATTACATTA
TCCACTGGATAAAACAGACACCTGGACAGGGCCTGGAATGGATTGGAGCT
ATTTATCCAGGAAATGGTGATACTTCCTACAGTCAGAAGTTCAAAGACAA
GGCCACATTGACTGCAGACAGATCCTCCAGCACAGCCTACATGCAGCTCA
GCAGCCTGACATCTGAGGACTCTGCGGTCTATTACTGTGCAAGAGGGGAT
GCGGGTTACTCCGCCTGGTTTGCTTACTGGGGCCAAGGGACTCTGGTCAC
TGTCTCTGCA

The amino acid sequence of light chain variable region of 49F10H6: SEQ ID NO: 19

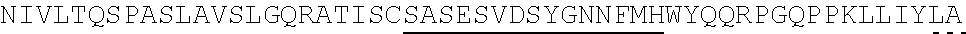

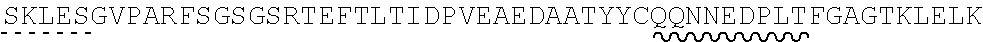

The nucleotide sequence of light chain variable region of 49F10H6: SEQ ID NO: 20

AACATTGTACTGACCCAATCTCCAGCTTCTTTGGCTGTGTCTCTAGGACA
GAGGGCCACCATATCCTGCAGTGCCAGTGAAAGTGTTGATAGTTATGGCA
ATAATTTTATGCACTGGTATCAGCAGAGACCAGGACAGCCACCCAAACTC
CTCATTTATCTTGCATCCAAGCTAGAATCTGGGGTCCCTGCCAGGTTCAG
TGGCAGTGGGTCTAGGACAGAGTTCACCCTCACCATTGACCCTGTGGAGG
CTGAAGATGCTGCAACCTATTACTGTCAGCAAAATAATGAGGATCCGCTC
ACGTTCGGTGCTGGGACCAAGCTGGAGCTGAAA

The amino acid sequence of heavy chain variable region of 53A2F9: SEQ ID NO: 21

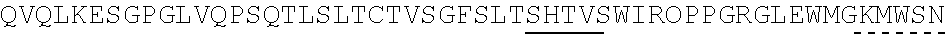

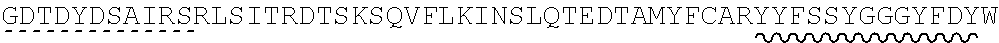

GQGVMVTVSS

The nucleotide sequence of heavy chain variable region of 53A2F9: SEQ ID NO: 22

CAAGTCCAACTAAAGGAGTCAGGACCTGGTCTGGTACAGCCATCACAGAC
CCTGTCTCTCACCTGCACTGTCTCTGGGTTTTCATTAACCAGCCATACTG
TAAGCTGGATTCGGCAGCCTCCAGGAAGGGGTCTGGAGTGGATGGGAAAA
ATGTGGAGTAATGGAGACACAGATTATGATTCAGCTATCAGATCCCGACT
GAGCATCACCAGGGACACCTCGAAGAGCCAAGTTTTCTTAAAGATAAACA
GTCTGCAAACTGAAGACACAGCCATGTACTTCTGTGCCAGATATTACTTT
AGCAGCTATGGAGGTGGCTACTTTGATTACTGGGGCCAAGGAGTCATGGT
CACAGTCTCCTCA

The amino acid sequence of light chain variable region of 53A2F9: SEQ ID NO: 23

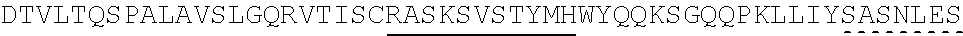

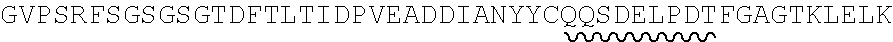

The nucleotide sequence of light chain variable region of 53A2F9: SEQ ID NO: 24

GACACTGTGCTGACCCAGTCTCCTGCTTTGGCTGTGTCTCTAGGACAGAG
GGTCACCATCTCTTGTAGGGCCAGCAAAAGTGTCAGTACATATATGCACT
GGTACCAACAGAAATCGGGACAGCAACCCAAACTCCTGATCTATAGTGCA
TCCAACCTAGAATCTGGAGTCCCTTCCAGGTTCAGTGGGAGTGGGTCTGG
GACAGACTTTACCCTCACCATAGATCCTGTGGAGGCTGATGACATAGCAA
ACTATTACTGTCAGCAGAGTGATGAACTTCCGGACACCTTTGGAGCTGGG
ACCAAGCTGGAACTGAAA

The amino acid sequence of heavy chain variable region of 61G1D8: SEQ ID NO: 25

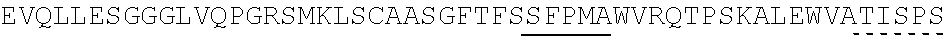

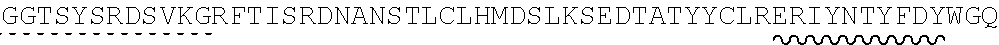

GVMVTVSS

The nucleotide sequence of heavy chain variable region of 61G1D8: SEQ ID NO: 26

GAGGTGCAGCTGCTAGAATCTGGGGGCGGTTTAGTGCAGCCTGGAAGGTC
CATGAAACTCTCCTGTGCGGCCTCAGGATTCACTTTCAGTAGCTTTCCAA
TGGCCTGGGTCCGCCAGACTCCATCGAAGGCTCTGGAGTGGGTCGCAACC
ATTAGTCCTAGTGGTGGTACCTCTTACTCTCGAGACTCCGTGAAGGGCCG
ATTCACTATCTCCAGAGATAATGCAAACAGCACCCTATGTCTACACATGG
ACAGTCTGAAGTCTGAGGACACGGCCACTTATTATTGTTTACGAGAAAGG
ATTTATAACACTTACTTTGATTACTGGGGCCAAGGAGTCATGGTCACAGT
CTCCTCA

The amino acid sequence of light chain variable region of 61G1D8: SEQ ID NO: 27

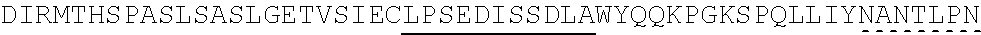

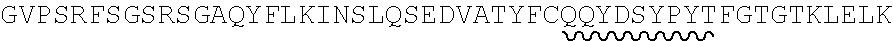

The nucleotide sequence of light chain variable region of 61G1D8: SEQ ID NO: 28

GATATCCGGATGACACACTCTCCAGCTTCCCTGTCTGCATCTCTGGGAGA
GACTGTCAGCATCGAATGTCTACCAAGTGAGGACATTTCCAGTGATTTAG
CATGGTATCAGCAGAAGCCAGGGAAATCTCCTCAGCTCCTCATCTATAAT
GCAAATACTTTGCCAAATGGGGTCCCTTCACGGTTTAGTGGCAGTAGATC
TGGCGCACAGTATTTTCTAAAAATAAACAGCCTGCAATCTGAGGATGTCG
CGACTTATTTCTGTCAGCAATATGACAGTTATCCGTACACGTTTGGAACT
GGGACCAAGCTGGAACTGAAA

DNA fragment comprising light chain variable region+constant region and DNA fragment comprising heavy chain variable region+constant region were synthesized, respectively, and were inserted into pTT5 expression vectors to form expression plasmids, respectively.

HEK293-6E cells were co-transfected with the expression plasmids above and cultured in a shake flask at 37° C. for 10 days. The supernatant was collected for antibody purification. Prior to purification, the tube and protein A column were depyrogenated with 0.2 M NaOH. The column was re-equilibrated with a buffer containing 0.05 M Tris and 1.5 M NaCl (pH 8.0). The harvested cell culture supernatant was then 1:1 diluted with 2× the buffer above and sterilized by filtration. The filtered supernatant and the protein A column were incubated at room temperature for 2 h. After washing the column with 1× the buffer above, IgG was eluted with sterile 0.1 M sodium citrate (pH 3.5). The eluate was collected and neutralized with one-ninth volume of sterile 1M Tris-HCl (pH9). Under sterile conditions, the buffer of the product was exchanged for PBS (pH7.4) to remove any elution buffer and the sample was concentrated. After concentration, antibodies were quantified by OD280 nm using an extinction coefficient Ec of 1.43 (0.1%).

Purified antibodies were analyzed by SDS-PAGE using 10% precast gel (GenScript) in BioRad electrophoresis system. The gel was stained with Estin2.0 (GenScript), and the molecular size and purity were estimated by comparing the staining bands with Protein Ladder (GenScript).

### Example 3: Binding of Monoclonal Antibodies to Human IL6 Recombinant Protein

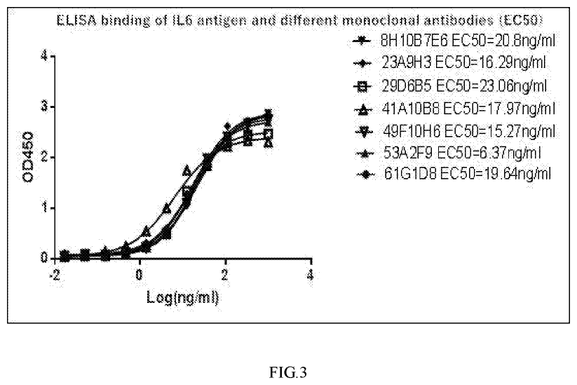

The purified antibodies were evaluated for the binding ability to IL6 by indirect ELISA. The ELISA plate (Nunc) was coated with 0.5 μg/ml recombinant IL6 protein in PBS at 100 μl/well at 4° C. overnight. The plate was washed with PBS-T (0.05% Tween) and was blocked with PBST containing 1% BSA at 200 μl/well at 37° C. for 0.5 h. The blocking solution was then discarded. 100 μl 10 μg/ml purified antibody was added to the first well, and 3-fold gradient dilution was performed for 11 test concentration gradients in total. Incubation was then performed at room temperature for 1 h. The plate was washed with PBST for three times and incubated with horseradish peroxidase-conjugated goat anti-mouse/rat IgG (Fab-specific) (GenScript) at 100 μl/well at 37° C. for 0.5 h. The plate was washed with PBST for five times, and TMB chromogenic solution (GenScript) was then added and incubation was performed in dark for 15 min at room temperature. The reaction was stopped by adding 50 μl 1M HCl stop solution (Sigma). The plate was read at 450 nm by a microplate reader. The binding ability of the clones 8H10B7E6, 23A9H3, 29D6B5, 41A10B8, 49F10H6, 53A2F9 (rat), and 61G1D8 (rat) for recombinant protein IL6 was shown in FIG. 3. EC50, as calculated according to ELISA concentration gradient experiments, showed that these antibodies exhibited relatively high affinity for the antigen (ELISA EC50 was less than 25 ng/ml; especially reached 6.32 ng/ml for 53A2F9, which was equivalent to 0.04 nM).

### Example 4: Identification of the Epitopes of Monoclonal Antibodies

The antigen epitopes of the purified antibodies were evaluated by competitive ELISA. The ELISA plate (Nunc) was coated with 0.5 μg/ml recombinant IL6 protein in PBS at 100 μl/well at 4° C. overnight. The plate was washed with PBS-T (0.05% Tween) and was blocked with PBST containing 1% BSA at 200 μl/well at 37° C. for 0.5 h. The blocking solution was then discarded. A pair of antibodies to be tested (one of which was biotin-labeled) for competitive experiment were added to each well at 100 μl (10 μg/ml) for each purified antibody. Incubation was then performed at 37° C. for 1 h. The plate was washed with PBST for three times and incubated with streptavidin HRP (SA-HRP, GenScript) at 100 μl/well at 37° C. for 10 min. The plate was washed with PBST for five times, and TMB chromogenic solution (GenScript) was then added and incubation was performed in dark for 15 min at room temperature. The reaction was stopped by adding 50 μl 1M HCl stop solution (Sigma). The plate was read at 450 nm by a microplate reader. The measurement results show that the clones 8H10B7E6, 23A9H3, 29D6B5 are directed against the same epitope, while 41A10B8, 49F10H6, 53A2F9 (rat), and 61G1D8 (rat) are directed against other four different epitopes, respectively. These results show that the obtained antibodies specific for IL6 have multiple different antigen recognition epitopes. Therefore, the antibodies screened out in the present invention have higher diversity.

### Example 5: Detection of the Functions of Monoclonal Antibodies

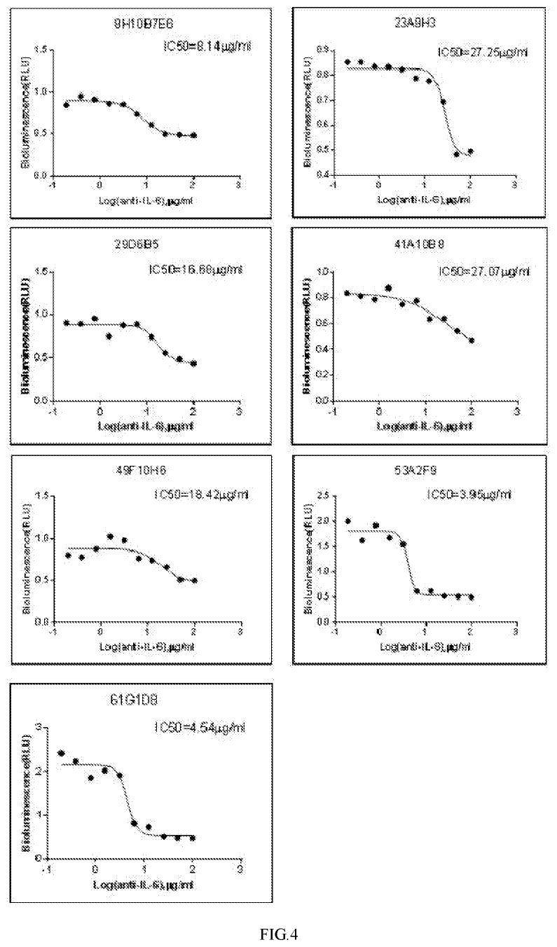

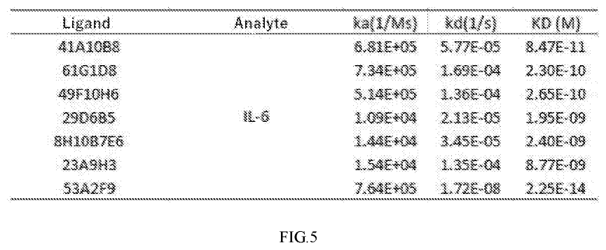

The growth of TF-1 cells is granulocyte macrophage colony-stimulating factor (GM-CSF)-dependent. If this factor is deficient or its function is blocked, the proliferation, differentiation and maturation of the cells will be significantly suppressed. In this monoclonal antibody function experiment, the GM-CSF in the medium was replaced with IL6 protein having the same function, and then anti-IL6 monoclonal antibodies were added to determine whether the monoclonal antibodies could block the biological function of IL6 protein by determining the number of live TF-1 cells after a certain period of culture. TF-1 cells were normally cultured in a medium containing GM-CSF, and the cells were collected prior to the experiment, washed twice with a basal medium without GM-CSF and counted. The cells were plated onto several wells in a 96-well plate at 5,000 cells/well (100 μl). IL6 protein was diluted to 0.4 μg/ml with the basal medium, and was added at 50 μl per well, wherein the protein content per well was 20 ng. The antibody was on another plate, and the dilution was started from 100 μg/ml in a 2-fold manner for 10 gradients. Finally, 50 μl was added to the well. Incubation was performed at 37° C. in 5% CO₂ for 7-10 days. 20 μl Promega Substrate Cell Titer 96 Aqueous One Solution Reagent was added to each well. Incubation was performed at 37° C., and the plate was read at 490 nm every half an hour. The best linear data was selected and edited. The inhibitory effect of IL6 antibodies on the growth of TF-1 cells was shown in FIG. 4. These results show that the antibodies screened out by the present invention could effectively inhibit cell proliferation, and especially the inhibitory IC50 reached 3.95 μg/ml, and 4.54 μg/ml for the clones 53A2F9 and 61G1D8 antibodies, respectively.

### Example 6: Measurement of Affinity of Monoclonal Antibodies

The chip surface was equilibrated with HBS-EP buffer at a flow rate of 10 μl/min for 5 min, and then the chip was activated by injecting a 1:1 mixture of “NHS+EDC” at a flow rate of 10 μl/min for 7 min. The capture antibody (Goat anti-mouse IgG) diluted in 10 mM sodium acetate buffer was injected at a flow rate of 10 μl/min for about 7 min for coupling, and finally ethanolamine was injected at a flow rate of 10 μl/min for 7 min for surface blocking.

HBS-EP buffer was used as a sample to perform three pre-cycles to equilibrate the chip so as to make the baseline stable, and the antibodies diluted in HBS-EP buffer was injected at a flow rate of 10 μl/min for 0 to 5 min (the binding signal of the antibody and the antigen was controlled at ˜100RU by adjusting the capture time), and the chip was equilibrated with buffer for 1 min. The low-concentration antigen 0.33 nM IL6 was injected at a flow rate of 30 μl/min for 5 min to enable the binding between the antigen and the antibody, and then the buffer was injected at a flow rate of 30 μl/min for 15 min to perform dissociation. 50 mM HCl was injected at a flow rate of 100 μl/min for four times and 10s each time for regeneration, and one cycle was completed. The antigen concentration (such as ln IL6) was changed for another cycle of determination at the next gradient concentration until the determination was completed for all the gradient concentrations (0.33 nM, 1 nM, 3 nM, 9 nM, 27 nM IL6) and repeated concentrations (e.g. 9 nM IL6).

After double deduction of experimental data (control channel and zero concentration), the fitting of a “1:1 Binding” model was performed in Biacore 8K evaluation software. Biacore 8K was used to determine the affinity of antibodies for IL recombinant protein. As shown in FIG. 5, the affinity of monoclonal antibodies specific for IL6 as measured by Biacore reached the magnitude of sub-nM to pM. These results show that the antibodies screened out by the present invention have a very high affinity.

It should be understood by those skilled in the art to which the present invention pertains that the methods and materials described above are merely exemplary and should not be considered as limiting the scope of the present invention.

1. An anti-human IL6 monoclonal antibody, comprising a heavy chain variable region and a light chain variable region, wherein the heavy chain variable region comprises HCDR1, HCDR2 and HCDR3, and the light chain variable region comprises LCDR1, LCDR2 and LCDR3, wherein the HCDR1, HCDR2, HCDR3, LCDR1, LCDR2 and LCDR3 are selected from one of the following combinations:
(a) the amino acid sequence of HCDR1 is RYWMH;
the amino acid sequence of HCDR2 is YINPITGYTENNQKFKD;
the amino acid sequence of HCDR3 is GIRGFTY;
the amino acid sequence of LCDR1 is RASENVDNSDNSFMH;
the amino acid sequence of LCDR2 is RASNLDS;
the amino acid sequence of LCDR3 is QQTNEAPLT;
(b) the amino acid sequence of HCDR1 is NYWMH;
the amino acid sequence of HCDR2 is YVIPSTGYTDYNQSFKD;
the amino acid sequence of HCDR3 is LLPGFAY;
the amino acid sequence of LCDR1 is RSSQSLVDSNGNTYLH;
the amino acid sequence of LCDR2 is KVSNRFS;
the amino acid sequence of LCDR3 is SQSTHVPPT;
(c) the amino acid sequence of HCDR1 is NYWMH;
the amino acid sequence of HCDR2 is YIDPRTASIYYNQKFKD;
the amino acid sequence of HCDR3 is ILYGKYDV;
the amino acid sequence of LCDR1 is RSSQSLVDSNGNTYLH;
the amino acid sequence of LCDR2 is KVSNRFS;
the amino acid sequence of LCDR3 is SQSTHVPPT;
(d) the amino acid sequence of HCDR1 is DAWMD;
the amino acid sequence of HCDR2 is EIRSKTYHPATYYTKSVRG;
the amino acid sequence of HCDR3 is PRYYGGYFDY;
the amino acid sequence of LCDR1 is RASESVDNYGMSFMN;
the amino acid sequence of LCDR2 is TASNQGS;
the amino acid sequence of LCDR3 is QQSKEVPYT;
(e) the amino acid sequence of HCDR1 is NYIIH;
the amino acid sequence of HCDR2 is AIYPGNGDTSYSQKFKD;
the amino acid sequence of HCDR3 is GDAGYSAWFAY;
the amino acid sequence of LCDR1 is SASESVDSYGNNFMH;
the amino acid sequence of LCDR2 is LASKLES;
the amino acid sequence of LCDR3 is QQNNEDPLT;
(f) the amino acid sequence of HCDR1 is SHTVS;
the amino acid sequence of HCDR2 is KMWSNGDTDYDSAIRS;
the amino acid sequence of HCDR3 is YYFSSYGGGYFDY;
the amino acid sequence of LCDR1 is RASKSVSTYMH;
the amino acid sequence of LCDR2 is SASNLES;
the amino acid sequence of LCDR3 is QQSDELPDT;
and
(g) the amino acid sequence of HCDR1 is SFPMA;
the amino acid sequence of HCDR2 is TISPSGGTSYSRDSVKG;
the amino acid sequence of HCDR3 is ERIYNTYFDY;
the amino acid sequence of LCDR1 is LPSEDISSDLA;
the amino acid sequence of LCDR2 is NANTLPN;
the amino acid sequence of LCDR3 is QQYDSYPYT.

2. The anti-human IL6 monoclonal antibody according to claim 1, wherein the amino acid sequences of its heavy chain variable region and light chain variable region are selected from one of the following combinations:
the heavy chain variable region comprising the amino acid sequence set forth in SEQ ID NO: 1, and the light chain variable region comprising the amino acid sequence set forth in SEQ ID NO: 3;
the heavy chain variable region comprising the amino acid sequence set forth in SEQ ID NO: 5, and the light chain variable region comprising the amino acid sequence set forth in SEQ ID NO: 7;
the heavy chain variable region comprising the amino acid sequence set forth in SEQ ID NO: 9, and the light chain variable region comprising the amino acid sequence set forth in SEQ ID NO: 11;
the heavy chain variable region comprising the amino acid sequence set forth in SEQ ID NO: 13, and the light chain variable region comprising the amino acid sequence set forth in SEQ ID NO: 15;
the heavy chain variable region comprising the amino acid sequence set forth in SEQ ID NO: 17, and the light chain variable region comprising the amino acid sequence set forth in SEQ ID NO: 19;
the heavy chain variable region comprising the amino acid sequence set forth in SEQ ID NO: 21, and the light chain variable region comprising the amino acid sequence set forth in SEQ ID NO: 23; and
the heavy chain variable region comprising the amino acid sequence set forth in SEQ ID NO: 25, and the light chain variable region comprising the amino acid sequence set forth in SEQ ID NO: 27.

3. The anti-human IL6 monoclonal antibody according to claim 1, wherein the dissociation constant KD between the antibody and IL6 is less than 10 nM.

4. The anti-human IL6 monoclonal antibody according to claim 1, wherein the dissociation constant KD between the antibody and IL6 is less than 1 nM.

5. The anti-human IL6 monoclonal antibody according to claim 1 or 2, wherein the antibody inhibits the promotion of TF-1 cell proliferation by IL6.

6-11. (canceled)

12. the anti-human IL6 monoclonal antibody according to claim 1 to the subject.

13. The method according to claim 12, wherein the tumor is selected from multiple myeloma, non-small cell lung cancer, colorectal cancer, renal cancer, prostate cancer, breast cancer and ovarian cancer.

14. An antitumor pharmaceutical composition, comprising an effective amount of the anti-human IL6 monoclonal antibody according to any claim 1 and a pharmaceutically acceptable carrier.

15. (canceled)

16. The method according to claim 12, wherein the the amino acid sequences of the heavy chain variable region and light chain variable region of the anti-human IL6 monoclonal antibody are selected from one of the following combinations:
the heavy chain variable region comprising the amino acid sequence set forth in SEQ ID NO: 1, and the light chain variable region comprising the amino acid sequence set forth in SEQ ID NO: 3;
the heavy chain variable region comprising the amino acid sequence set forth in SEQ ID NO: 5, and the light chain variable region comprising the amino acid sequence set forth in SEQ ID NO: 7;
the heavy chain variable region comprising the amino acid sequence set forth in SEQ ID NO: 9, and the light chain variable region comprising the amino acid sequence set forth in SEQ ID NO: 11;
the heavy chain variable region comprising the amino acid sequence set forth in SEQ ID NO: 13, and the light chain variable region comprising the amino acid sequence set forth in SEQ ID NO: 15;
the heavy chain variable region comprising the amino acid sequence set forth in SEQ ID NO: 17, and the light chain variable region comprising the amino acid sequence set forth in SEQ ID NO: 19;
the heavy chain variable region comprising the amino acid sequence set forth in SEQ ID NO: 21, and the light chain variable region comprising the amino acid sequence set forth in SEQ ID NO: 23; and
the heavy chain variable region comprising the amino acid sequence set forth in SEQ ID NO: 25, and the light chain variable region comprising the amino acid sequence set forth in SEQ ID NO: 27.

17. The method according to claim 12, wherein the dissociation constant KD between the anti-human IL6 monoclonal antibody and IL6 is less than 10 nM.

18. The method according to claim 12, wherein the dissociation constant KD between the anti-human IL6 monoclonal antibody and IL6 is less than 1 nM.

19. The method according to claim 12, wherein the anti-human IL6 monoclonal antibody inhibits the promotion of TF-1 cell proliferation by IL6.

20. The antitumor pharmaceutical composition according to claim 14, wherein the the amino acid sequences of the heavy chain variable region and light chain variable region of the anti-human IL6 monoclonal antibody are selected from one of the following combinations:
the heavy chain variable region comprising the amino acid sequence set forth in SEQ ID NO: 1, and the light chain variable region comprising the amino acid sequence set forth in SEQ ID NO: 3;
the heavy chain variable region comprising the amino acid sequence set forth in SEQ ID NO: 5, and the light chain variable region comprising the amino acid sequence set forth in SEQ ID NO: 7;
the heavy chain variable region comprising the amino acid sequence set forth in SEQ ID NO: 9, and the light chain variable region comprising the amino acid sequence set forth in SEQ ID NO: 11;
the heavy chain variable region comprising the amino acid sequence set forth in SEQ ID NO: 13, and the light chain variable region comprising the amino acid sequence set forth in SEQ ID NO: 15;
the heavy chain variable region comprising the amino acid sequence set forth in SEQ ID NO: 17, and the light chain variable region comprising the amino acid sequence set forth in SEQ ID NO: 19;
the heavy chain variable region comprising the amino acid sequence set forth in SEQ ID NO: 21, and the light chain variable region comprising the amino acid sequence set forth in SEQ ID NO: 23; and
the heavy chain variable region comprising the amino acid sequence set forth in SEQ ID NO: 25, and the light chain variable region comprising the amino acid sequence set forth in SEQ ID NO: 27.

21. The antitumor pharmaceutical composition according to claim 14, wherein the dissociation constant KD between the anti-human IL6 monoclonal antibody and IL6 is less than 10 nM.

22. The antitumor pharmaceutical composition according to claim 14, wherein the dissociation constant KD between the anti-human IL6 monoclonal antibody and IL6 is less than 1 nM.

23. The antitumor pharmaceutical composition according to claim 14, wherein the anti-human IL6 monoclonal antibody inhibits the promotion of TF-1 cell proliferation by IL6.


SEQUENCE LISTING (28 sequences)

SEQ ID NO: 1 - protein, 100 aa, Artificial Sequence
  Synthetic Construct
SVKMSCKASGYTLTRYWMHWVKQRPGQGLEWIGYINPITGYTENNQKFKDKATLTADKSSSTAYIQLSSLTSEDSAVYFCARGIRGFTYWGHGTLVTVSA

SEQ ID NO: 2 - DNA, 348 bp, Artificial Sequence
  Synthetic Construct
caggtccagcttcagcagtctggggctgaactggcaaaacctggggcctcagtgaagatgtcctgcaaggcttctggctacaccttaactaggtactggatgcactgggtaaaacagaggcctggacagggtctggaatggattggatacattaatcctatcactggttatactgagaacaatcagaagttcaaggacaaggccacattgactgcagacaagtcctccagcacagcctatatacaactgagcagcctgacatctgaggactctgcagtctatttctgtgcaagagggatacgggggtttacttactggggccatgggactctggtcactgtctctgca

SEQ ID NO: 3 - protein, 111 aa, Artificial Sequence
  Synthetic Construct
DIVLTQSPASLAVSLGQRATISCRASENVDNSDNSFMHWYQQKPGQPPKLLIYRASNLDSGIPARFSGSGSRTDFTLTINPVEADDVATYYCQQTNEAPLTFGAGTKLELK

SEQ ID NO: 4 - DNA, 333 bp, Artificial Sequence
  Synthetic Construct
gacattgtgctgacccaatctccagcttctttggctgtgtctctagggcagagggccaccatatcctgcagagccagtgagaatgttgataactctgacaatagttttatgcactggtaccaacagaaaccaggacagccacccaaactcctcatctatcgtgcatccaacctagattctgggatccctgccaggttcagtggcagtgggtctaggacagacttcaccctcaccattaatcctgtggaggctgatgatgttgcaacctattactgtcagcaaactaatgaggctccgctcacgttcggtgctgggaccaagctggagctgaaa

SEQ ID NO: 5 - protein, 116 aa, Artificial Sequence
  Synthetic Construct
QVQLQQSGAELAKPGASVKMSCKTSGYTFTNYWMHWVKQRPGQGLEWIGYVIPSTGYTDYNQSFKDKATLTADKSSNTAYMQVNSLTSEDSAVYYCASLLPGFAYWGQGTLVTVSA

SEQ ID NO: 6 - DNA, 348 bp, Artificial Sequence
  Synthetic Construct
caggtccagcttcagcagtctggggctgaactggcaaaacctggggcctcagtgaagatgtcctgcaagacttctggctacacctttactaactactggatgcactgggtaaaacagaggcctggacagggtctggagtggattggatacgttattcctagcactggttatactgactacaatcagagtttcaaggacaaggccacattgactgcagacaaatcctccaacacagcctacatgcaagtgaacagcctgacatctgaggactctgcagtctattactgtgcaagcctactgccaggttttgcttactggggccaagggactctggtcactgtctctgca

SEQ ID NO: 7 - protein, 112 aa, Artificial Sequence
  Synthetic Construct
DVVMTQTPLSLPVSLGDQASISCRSSQSLVDSNGNTYLHWYLQKPGQSPKLLIYKVSNRFSGVPDRFSGSGSGTDFTLKISRVEAEDLGVYFCSQSTHVPPTFGGGTKLEIK

SEQ ID NO: 8 - DNA, 336 bp, Artificial Sequence
  Synthetic Construct
gatgttgtgatgacccaaactccactctccctgcctgtcagtcttggagatcaagcctccatctcttgcagatctagtcagagccttgtggacagtaatggaaacacttatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggggtttatttctgctctcaaagtacacatgttcctccgacgttcggtggaggcaccaagctggaaatcaaa

SEQ ID NO: 9 - protein, 117 aa, Artificial Sequence
  Synthetic Construct
QVQLQQSGAELAEPGASVKMSCKASGYNFNNYWMHWVKQGPGQGLEWIGYIDPRTASIYYNQKFKDKATLTADKSSSTAYMQLSSLTSEDSAVYYCARILYGKYDVWGAGTTVIVSS

SEQ ID NO: 10 - DNA, 351 bp, Artificial Sequence
  Synthetic Construct
caggtccagcttcagcagtctggggctgaactggcagaacctggggcctcagtgaagatgtcctgcaaggcttctggctacaactttaataactattggatgcactgggtaaaacaggggcctggccagggtctggaatggattggatacattgatcctaggactgcttctatttattacaatcagaagttcaaagacaaggccacattgactgcagacaaatcctccagcacagcctacatgcaactgagcagcctgacatctgaagactctgcagtctattactgtgcaagaatcctctatggtaaatatgatgtctggggcgcagggaccacggtcatcgtctcctca

SEQ ID NO: 11 - protein, 112 aa, Artificial Sequence
  Synthetic Construct
DVVLTQTPLSLPVSLGNQASISCRSSQSLVDSNGNTYLHWYLQKPGQSPKVLIHKVSNRFSGVPDRFSGSGSGTDFTLKISRVEAEDLGVYFCSQSTHVPPTFGGGTKLEIK

SEQ ID NO: 12 - DNA, 336 bp, Artificial Sequence
  Synthetic Construct
gatgttgtgctgacccaaactccactctccctgcctgtcagtcttggaaatcaagcctccatctcttgcagatctagtcagagccttgtagacagtaatggaaacacctatttacattggtacctgcagaagccaggccagtctccaaaggtcctgatccacaaagtttccaaccgattttctggggtcccagacaggttcagtggcagtggatcagggacagatttcacactcaagatcagcagagtggaggctgaggatctgggagtttatttctgctctcaaagtacacatgttcctccgacgttcggtggaggcaccaagctggaaatcaaa

SEQ ID NO: 13 - protein, 121 aa, Artificial Sequence
  Synthetic Construct
EVKLEESGGGLVQPGGSMKLSCAASGFTFSDAWMDWVRQSPDKGLEWVAEIRSKTYHPATYYTKSVRGRFTISRDDSKSSVYLQMNNLGAEDTGIYYCTRPRYYGGYFDYWGQGTTLTVSS

SEQ ID NO: 14 - DNA, 363 bp, Artificial Sequence
  Synthetic Construct
gaagtgaaacttgaggagtctggaggaggcttggtgcaacctggaggatccatgaaactctcttgtgctgcctctggattcacttttagtgacgcctggatggactgggtccgccagtctccagacaaggggcttgagtgggttgctgaaattagaagcaaaacttatcatcctgcaacatactatactaagtctgtgagagggaggttcaccatctcaagagatgattccaaaagtagtgtctacctgcagatgaacaacttaggagctgaagacactggcatttattactgtaccaggccgagatactacgggggttactttgactactggggccaaggcaccactctcacagtctcctca

SEQ ID NO: 15 - protein, 111 aa, Artificial Sequence
  Synthetic Construct
DIVLTQSPASLAVSLGQRATISCRASESVDNYGMSFMNWFQQKPGQPPKLLIYTASNQGSGVPARFSGSGSGTDFSLNIHPMEEDDTAMYFCQQSKEVPYTFGGGTKLEIK

SEQ ID NO: 16 - DNA, 333 bp, Artificial Sequence
  Synthetic Construct
gacattgtgctgacccaatctccagcttctttggctgtgtctctagggcagagggccaccatctcctgcagagccagcgaaagtgttgacaattatggcatgagttttatgaactggttccaacagaaaccaggacagccacccaaactcctcatctatactgcttccaaccaaggatccggggtccctgccaggtttagtggcagtgggtctgggacagacttcagcctcaacatccatcctatggaggaggatgatactgcaatgtatttctgtcaacaaagtaaggaggtcccgtatacgttcggaggggggaccaagctggaaataaaa

SEQ ID NO: 17 - protein, 120 aa, Artificial Sequence
  Synthetic Construct
QVQLQQPGAELVKPGASVKMSCKASGYTFTNYIIHWIKQTPGQGLEWIGAIYPGNGDTSYSQKFKDKATLTADRSSSTAYMQLSSLTSEDSAVYYCARGDAGYSAWFAYWGQGTLVTVSA

SEQ ID NO: 18 - DNA, 360 bp, Artificial Sequence
  Synthetic Construct
caggtgcaactgcagcagcctggggctgaactggtgaagcctggggcctcagtgaagatgtcctgcaaggcttctggctacacatttaccaattacattatccactggataaaacagacacctggacagggcctggaatggattggagctatttatccaggaaatggtgatacttcctacagtcagaagttcaaagacaaggccacattgactgcagacagatcctccagcacagcctacatgcagctcagcagcctgacatctgaggactctgcggtctattactgtgcaagaggggatgcgggttactccgcctggtttgcttactggggccaagggactctggtcactgtctctgca

SEQ ID NO: 19 - protein, 111 aa, Artificial Sequence
  Synthetic Construct
NIVLTQSPASLAVSLGQRATISCSASESVDSYGNNFMHWYQQRPGQPPKLLIYLASKLESGVPARFSGSGSRTEFTLTIDPVEAEDAATYYCQQNNEDPLTFGAGTKLELK

SEQ ID NO: 20 - DNA, 333 bp, Artificial Sequence
  Synthetic Construct
aacattgtactgacccaatctccagcttctttggctgtgtctctaggacagagggccaccatatcctgcagtgccagtgaaagtgttgatagttatggcaataattttatgcactggtatcagcagagaccaggacagccacccaaactcctcatttatcttgcatccaagctagaatctggggtccctgccaggttcagtggcagtgggtctaggacagagttcaccctcaccattgaccctgtggaggctgaagatgctgcaacctattactgtcagcaaaataatgaggatccgctcacgttcggtgctgggaccaagctggagctgaaa

SEQ ID NO: 21 - protein, 121 aa, Artificial Sequence
  Synthetic Construct
QVQLKESGPGLVQPSQTLSLTCTVSGFSLTSHTVSWIRQPPGRGLEWMGKMWSNGDTDYDSAIRSRLSITRDTSKSQVFLKINSLQTEDTAMYFCARYYFSSYGGGYFDYWGQGVMVTVSS

SEQ ID NO: 22 - DNA, 363 bp, Artificial Sequence
  Synthetic Construct
caagtccaactaaaggagtcaggacctggtctggtacagccatcacagaccctgtctctcacctgcactgtctctgggttttcattaaccagccatactgtaagctggattcggcagcctccaggaaggggtctggagtggatgggaaaaatgtggagtaatggagacacagattatgattcagctatcagatcccgactgagcatcaccagggacacctcgaagagccaagttttcttaaagataaacagtctgcaaactgaagacacagccatgtacttctgtgccagatattactttagcagctatggaggtggctactttgattactggggccaaggagtcatggtcacagtctcctca

SEQ ID NO: 23 - protein, 106 aa, Artificial Sequence
  Synthetic Construct
DTVLTQSPALAVSLGQRVTISCRASKSVSTYMHWYQQKSGQQPKLLIYSASNLESGVPSRFSGSGSGTDFTLTIDPVEADDIANYYCQQSDELPDTFGAGTKLELK

SEQ ID NO: 24 - DNA, 318 bp, Artificial Sequence
  Synthetic Construct
gacactgtgctgacccagtctcctgctttggctgtgtctctaggacagagggtcaccatctcttgtagggccagcaaaagtgtcagtacatatatgcactggtaccaacagaaatcgggacagcaacccaaactcctgatctatagtgcatccaacctagaatctggagtcccttccaggttcagtgggagtgggtctgggacagactttaccctcaccatagatcctgtggaggctgatgacatagcaaactattactgtcagcagagtgatgaacttccggacacctttggagctgggaccaagctggaactgaaa

SEQ ID NO: 25 - protein, 119 aa, Artificial Sequence
  Synthetic Construct
EVQLLESGGGLVQPGRSMKLSCAASGFTFSSFPMAWVRQTPSKALEWVATISPSGGTSYSRDSVKGRFTISRDNANSTLCLHMDSLKSEDTATYYCLRERIYNTYFDYWGQGVMVTVSS

SEQ ID NO: 26 - DNA, 357 bp, Artificial Sequence
  Synthetic Construct
gaggtgcagctgctagaatctgggggcggtttagtgcagcctggaaggtccatgaaactctcctgtgcggcctcaggattcactttcagtagctttccaatggcctgggtccgccagactccatcgaaggctctggagtgggtcgcaaccattagtcctagtggtggtacctcttactctcgagactccgtgaagggccgattcactatctccagagataatgcaaacagcaccctatgtctacacatggacagtctgaagtctgaggacacggccacttattattgtttacgagaaaggatttataacacttactttgattactggggccaaggagtcatggtcacagtctcctca

SEQ ID NO: 27 - protein, 107 aa, Artificial Sequence
  Synthetic Construct
DIRMTHSPASLSASLGETVSIECLPSEDISSDLAWYQQKPGKSPQLLIYNANTLPNGVPSRFSGSRSGAQYFLKINSLQSEDVATYFCQQYDSYPYTFGTGTKLELK

SEQ ID NO: 28 - DNA, 321 bp, Artificial Sequence
  Synthetic Construct
gatatccggatgacacactctccagcttccctgtctgcatctctgggagagactgtcagcatcgaatgtctaccaagtgaggacatttccagtgatttagcatggtatcagcagaagccagggaaatctcctcagctcctcatctataatgcaaatactttgccaaatggggtcccttcacggtttagtggcagtagatctggcgcacagtattttctaaaaataaacagcctgcaatctgaggatgtcgcgacttatttctgtcagcaatatgacagttatccgtacacgtttggaactgggaccaagctggaactgaaa